(19) United States
(12) Patent Application Publication
(10) Pub. No.: US 2015/0132849 A1
(43) Pub. Date: May 14, 2015

(54) EX VIVO MATURATION OF ISLET CELLS

(71) Applicant: Jonathan Rt Lakey, Orange, CA (US)

(72) Inventor: Jonathan Rt Lakey, Orange, CA (US)

(73) Assignee: ISLET SCIENCES, INC., New York, NY (US)

(21) Appl. No.: 14/346,454

(22) PCT Filed: Sep. 28, 2012
(86) PCT No.: PCT/US12/58087

§ 371 Date: Mar. 21, 2014

Related U.S. Application Data
(60) Provisional application No. 61/540,288, filed on Sep. 28, 2011.
(60) Provisional application No. 61/540,293, filed on Sep. 28, 2011.

(51) Int. Cl. C12N 5/071

(52) U.S. Cl. CPC C12N 5/0676; C12N 2501/73; C12N 2501/734; C12N 2501/91; C12N 2501/335; C12N 2501/33; C12N 2500/38; C12N 2500/25
USPC 435/377

(57) ABSTRACT

The invention relates to methods for promoting maturation of islet cells from pre-weaned mammals for the purpose of optimizing the islets for their use as donor tissue for xenotransplantation. The method of the invention removes the pancreas from donor animals and reduces the pancreas tissue to fragments that are greater than the size of an intact islet while retaining islets in their whole, insulin-producing condition. The method of the invention also serially cultures the digested tissue in novel maturation media that enhance the glucose responsiveness of the cultured islets, and selects islets that are sufficiently glucose-responsive for use in transplantation procedures.

### CROSS-REFERENCE TO RELATED APPLICATIONS

Priority is claimed to U.S. Application Ser. No. 61/540,288 and to U.S. Application Ser. No. 61/540,293, which were filed 28 Sep. 2011, the entire disclosures of which are incorporated herein by reference.

### FIELD OF THE INVENTION

The invention relates to methods of isolating and culturing immature islets of Langerhans to maturity.

### BACKGROUND

Diabetes is a group of disorders with a number of common features, of which raised blood glucose is the most evident. The four commonest types of diabetes are Type 1 diabetes (T1D), Type 2 diabetes, secondary diabetes, and gestational diabetes. T1D is an Insulin-deficiency disease, developing predominantly in childhood, and characterized by hyperglycaemia if untreated. T2D is a disorder of insulin insensitivity coupled with a failure of pancreatic insulin secretion to compensate for the insulin insensitivity. Secondary diabetes develops subsequent to an insult to the endocrine system such as pancreatic damage, hepatic cirrhosis, endocrinological disorders, or certain anti-viral or anti-psychotic therapies. Gestational diabetes develops as a complication to pregnancy.

A body's only source of Insulin are the beta cells that reside in the Islets of Langerhans (hereafter, “islets”) of the pancreas. Islets are cytoarchitecturially-unique aggregates of endocrine cells that comprise approximately about 2% of the total volume of the Pancreas. In addition to insulin-producing beta cells, islets also contain alpha cells, which produce glucagon, delta cells, which produce somatostatin, PP cells, which produce pancreatic polypeptide, and epsilon cells, which produce ghrelin. Together, the cells of the islets regulate endocrine function, and treatments for metabolic disorders often involve restoring or modulating islet exocrine functions. One of these treatments is islet allotransplantation, and it can partially or totally cure T1D for periods of months to years. However, its therapeutic utilization remains limited because of the shortage of pancreas donors, which are the sole clinically-recognized source of human islets (Scharp D, et al. (1990) Diabetes. 39: 515-518; Wier G C, et al. (1997) Diabetes 1997; 46:1247; Shapiro A M, et al. (2000) N Engl J Med 2000; 343:2303; and Ichii H, et al. (2009) J Hepatobiliary Pancreat Surg. 16(2):101-12. Epub 2008 Dec. 26.

A potential remedy for the undersupply of human islets is the xenotransplantation of non-human islets that are encapsulated in immune response-protective barriers. See FIG. 1 and (Orive G, et al. (2003) Nat Med. 9:104; Duvivier-Kali V F, et al. (2001) Diabetes. 50:1698; and Schneider S, et al. (2005) Diabetes 54: 687). In particular, pigs are an appropriate source of islets because of their islets' similar size and physiology to those of humans, the nearly identical molecular structures of human and porcine insulin, and the long history of the safe use of porcine insulin in the treatment of human diabetes. Pigs also offer the additional advantages of having large litter sizes and being relatively easy to husband.

The isolation of mature porcine islets, i.e., fully functional islets, for use in transplants is currently only performed by using market-size or young adult pigs as donor animals. However, the isolation of islets from young adult and market weight pigs is labor-intensive and requires an extensive amount of equipment and supplies, including the resources that are required to raise the pigs to the appropriate size. The final yield of adult porcine islets, which are inherently fragile, is also sensitive to factors such as breed, pancreas quality, and the fact that the vast majority (around 98%) of the pancreatic tissue that surrounds islets is highly enzymatically active exocrine tissue that begins to destroy islets immediately upon the initiation of conventional islet isolation procedures. Indeed, the foregoing factors exacerbate the fragility of the islets and cause substantial islet fragmentation (i.e., disruption of their cytoarchitecture) during the isolation, storage, and culture of the islets (Socci C, et al. (1989) Horm. Metab. Res. 25(Suppl. 1):32-35; Kirchof N, et al. (1994) Transplant. Proc. 26:616-617; Van Deijnen J H M, et al. (1992) Cell Tissue Res. 267:139-146; and Marchetti P, et al. (1992) Diab. Nutr. Metab. 5(Suppl. 1): 151-154). Consequently, islet yield per pancreas is substantially lower than the theoretical yield based on an intrinsic islet count per pancreas. Tissue samples stained with H&E revealed young pig pancreases to be less dense and to have diffusely scattered insulin positive cells without well demarcated islet structures in contrast to the well defined islets found in the adult pig pancreas. See FIG. 2.

Neonatal and fetal pigs may also be used as an islet donors. An advantage that these islet sources offer is that the pancreases at these stages of development are immature, and thus, contain less exocrine tissue to damage islets during the islet isolation procedure. However, there are also immaturity-related disadvantages of using these islet sources, such as the islets' indistinct islet demarcations and their structural delicacy at these stages. Nonetheless, it is possible to isolate immature islets from neonatal and fetal pancreases (Korsgran 0, et al. (1988) Transplantation 1988; 45:509-14; Latif Z A, et al. (1988) Transplantation. 45(4):827-830; 1988; and Ricordi C, et al. (1990) Islet isolation assessment in man and large animals. Acta Diabetol Lat. 27(3):185-95. However, these immature islets are not capable of secreting insulin in response to glucose for at lease two weeks after their isolation, and may take as long as two to three months after being transplanted to a host to become glucose responsive (Korsgran 0, et al. (1988) Transplantation 1988; 45:509-14). Therefore, islets from neonatal and fetal pancreatic tissue are not clinically desirable because of their variable glucose responsiveness, and for the long period of time they take to mature.

As an alternative to the conventional islet transplantation approaches, which are problematic at least because of the reasons outlined above, the invention described herein is a reproducible, cost-effective, and scalable method (herein “the method of the invention”) for isolating and culturing islets that results in high yields of islet equivalents, i.e., islets that are glucose-responsive prior to transplantation. Thus, the method of the invention will enable informed insulin dosing decisions because the insulin secretory function of an islet transplant can be confirmed prior to transplantation.

### SUMMARY OF THE INVENTION

The invention relates to methods for promoting maturation of islet cells from pre-weaned mammals for the purpose of optimizing the islets for their use as donor tissue for xenotransplantation. In various embodiments, the donor animals are piglets. The method of the invention removes the pancreas from donor animals and reduces the pancreas tissue to fragments that are greater than the size of an intact islet while retaining islets in their whole, insulin-producing condition. The method of the invention also digests the resulting tissue fragments in a protease solution that gently separates exocrine tissue that surrounds the islets without causing extensive damage to the islet cells or the cytoarchitecture of the islets. The method of the invention also serially cultures the digested tissue in novel maturation media that enhance the glucose responsiveness of the cultured islets, and selects islets that are sufficiently glucose-responsive for use in transplantation procedures. In various embodiments of the invention, the maturation media comprise a) an undefined component of animal origin; b) an antioxidant compound; c) a component that enhances glucose-dependent insulin secretion by beta cells; d) a derivative of vitamin B; f) a derivative of vitamin E; g) heparin; h) a basic pancreatic trypsin inhibitor; i) a serine protease inhibitor; and j) a deoxyribonuclease.

### BRIEF DESCRIPTION OF THE FIGURES

FIG. 1 is a flow chart of the islet cell isolation procedure, including the sectioning of young porcine pancreas, treatment of the tissue sections with a collagenase mixture, and processing of the islets for xenotransplantation.

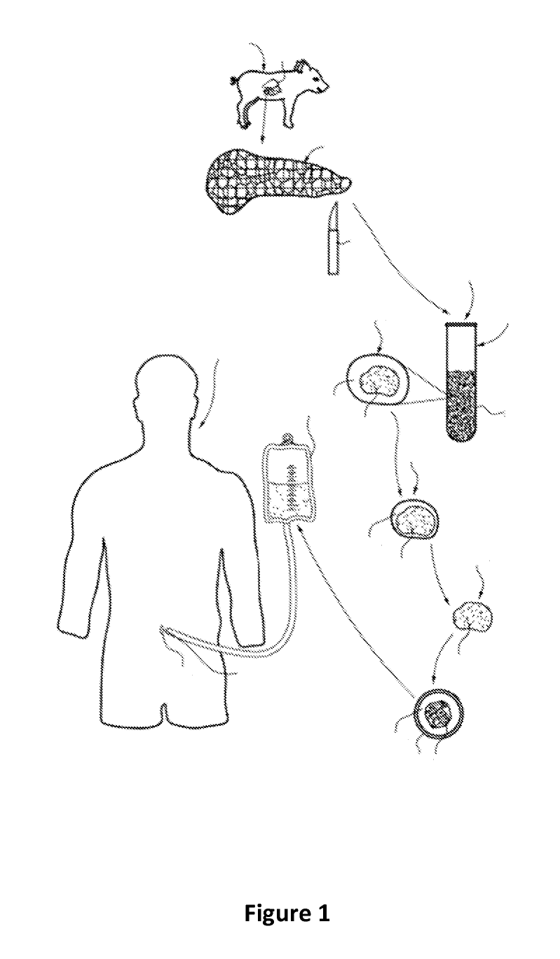

FIG. 2 exemplifies a histological comparison of adult porcine pancreas versus young porcine pancreas. Tissue samples were taken from splenic end of the pancreases from either market weight adult or young pigs (15 days old). Pancreas samples were fixed and either stained with hematoxylin and eosin stain (H&E) or anti-insulin and hematoxylin (Insulin). Adult pancreas showed complete islet formation, and an overall denser cell populations (denser volume of nuclei), while the young porcine pancreas showed no visible intact islet formations and unstructured insulin and glucagon staining when compared to adult porcine pancreas. All images taken at 20× magnification, Scale=100 μm.

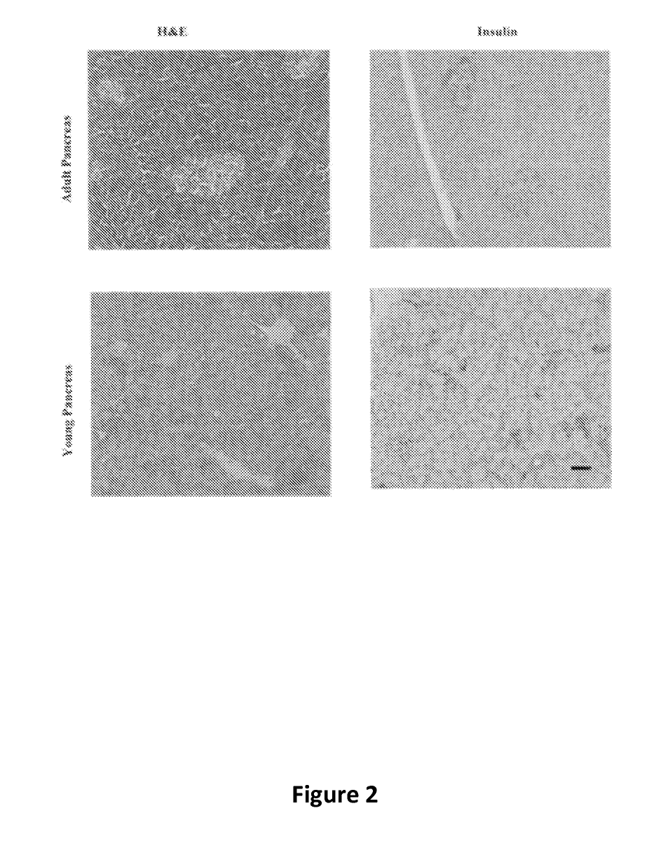

FIG. 3 shows changes in the number of islets, including islet equivalents, purity, viability and function over the maturation culture period. Islet samples were taken for analyses at 0, 1, 3, 5, 7, 9, and 14 days post digestion, during islet maturation. Islet tissue samples were stained with either dithizone (DTZ) to identify islet equivalents and islet purity, or fluorescein diacetate/propidium iodide (FDA/PI) to determine cell viability. Islet equivalents (A) were calculated as the percentage of positive DTZ staining. Islet purity (B, grey line) was calculated as percentage of unstained tissue, and islet viability (B, black line) was calculated as percentage alive (green)/percentage dead (red). For islet function (C), 100 islets were incubated at 37° C., at 5% CO₂, for an hour in 2.8 mM glucose, followed by 28 mM glucose, followed by 28 mM glucose plus IBMX, and then 2.8 mM glucose. Samples were analyzed for insulin release using a standard porcine insulin ELISA. In (D), an image of an islet sample that was stained with FDA/PI after 5 days of culture shows surrounding tissue is characterized primarily by cell death, while the internal compartment of the islet remains viable. Results are displayed as mean±SEM per pancreas, (n=6 in duplicate).

### BRIEF DESCRIPTION OF THE TABLES

Table 1 reports data that characterizes islet maturation over a 14 Day period in tissue culture. Islets were partially enzymatically dissociated and allowed to mature in tissue culture (37° C., 5% CO₂) for 14 days. Islet samples were taken directly following enzymatic digestion, after 3 days, 7 days and 14 days in culture and stained with Dithizone (DTZ) to quantify number of islets (islet count), and to determine the “islet equivalent,” (i.e., the percentage of stained versus unstained islets). The percentages of islet sizes was determined using a standard stereomicroscope equipped with a standard 10×10 eyepiece graticule (25× magnification, n=number if pancreases). Results displayed as mean±SEM.

Table 2 reports data that characterizes the cellular compositions of islets at various points over the course of their maturation in tissue culture. Islet samples were dissociated at 0, 3, 7, and 11 days during maturation and stained for either C-peptide, Glucagon, or Amylase and analyzed using Flow cytometry. Results are given as mean±SEM, n=3, performed in triplicate.

### DETAILED DESCRIPTION

The invention relates to methods for promoting the ex vivo maturation of pancreatic islets into glucose-responsive mature islets that can be used as donor tissue for xenotransplantation. It is to be understood herein that the method of the invention isolates and processes islets removed from the pancreas of a young mammal. For example, in various embodiments, the method of the invention isolates islets removed from the pancreas of a mammal that is at any stage of development between birth and being weaned, i.e., no longer nursing. The rationale for using pre-weaned donor pigs in the method of the invention is that consumption of solid food by a post-weaned pig activates the production of digestive enzymes by pancreatic exocrine tissue which, in turn, damage islets upon removal and processing of the pancreas from the animal. Thus, with the foregoing rationale in mind, there are also embodiments of the method of the invention that allow for the use of donor pancreases of animals that are of ages that are typically associated with being weaned, but have not yet begun to consume solid food.

In various embodiments, the method of the invention uses donor pancreases that are obtained from young pigs, i.e., piglets. Typically, such young pigs are about 30 days old or younger. In some embodiments, for example, the method of the invention isolates and cultures islets from piglets that are about 14 to about 22 days old, whereas in other embodiments, the young piglet islet donors may be about 18 to about 28 days old.

As stated above, the method of the invention isolates and processes islets from donor pancreatic tissue. It is to be understood herein, that the term “islets” as it used in the invention includes any cellular structures that are recognized by those of skill in the art as having the morphological and histological features of Islets of Langerhans, as well as includes less structured cellular aggregates that comprise beta cells. Islets, as disclosed herein, are advantageous, at least in part, because they possess certain morphological characteristics that are associated with successful xenotransplantation, and because the cells maintain an islet-like architecture that is associated with glucose responsiveness.

In various embodiments, the method of the invention surgically removes a mammalian pancreas, including the trimming away of any non-pancreatic tissue (e.g., fat, etc.). Generally, between ten and fifteen pancreases are harvested, and then combined for tissue processing. Processing pancreatic tissue involves using techniques that reduce the tissue to sections that are about the size of intact islets, while taking care not to disturb the cellular arrangement and architecture of the islets. Usually, the sectioning of the pancreatic tissue is performed under chilled conditions. In various embodiments, sectioning is performed by using scissors and blender technology. In various embodiments, the pancreatic tissue is sectioned into pieces that are, at their longest, from about 0.1 mm to about 10 mm. In certain other embodiments, the pancreatic tissue sections are, at their longest dimension, about 0.5 mm to about 1.0 mm in diameter. In general, the amount of time that elapses from removal of the pancreas to completion of the mincing step is about 20 minutes or less. However, in various embodiments, the method of the invention, the amount of time that elapses from removal of the pancreas to completion of the mincing step may be more than 20 minutes, for example, as long as 30 or 40 minutes.

The method of the invention also enzymatically digests sectioned, i.e., minced, pancreatic tissue to separate islets from non-islet tissue. In general, the method of the invention contacts the minced pancreatic tissue with a low-dose, preferably aseptic, collagenase solution. In various embodiments of the method of the invention, a collagenase solution may comprise about 60% purified class I (C1) and about 40% purified class II (C2) collagenase from Clostidium histolyticum. In various other embodiments, a collagenase solution can also comprise other types of proteases in addition to C1 and C2 collagenases. For example, the Collagenase solution may also comprise C. histolyticum collagenase Class 3 (C3), C. histolyticum collagenase Class 4 (C4), clostripain, thennalysin, trypsin, chymotrypsin, or elastase, or any combination thereof, e.g., like those combinations of proteases that are commercially marked as Liberase® and Blendzyme®. Enzymatic digestion of the sectioned pancreatic tissue is generally performed at 37° C. while gently rotating at 40-70 rpm until the majority of the acinar stromal tissue that surrounds islets separates from the islets. In various embodiments, the method of the invention continues the digestion reaction for an additional period of time under the same reaction conditions after the surrounding acinar tissue separates from the islet cells to allow the collagen matrices of the islets to be more completely digested. The length of time that an additional collagenase digestion period may take is not limited, but can take as long as one of skill in the art decides in necessary to remove tissue that is extraneous to the islets without excessively damaging the islets. In general, the method of the invention keeps the collagenase digestion as brief as possible to aid in the maintenance of nascent islet architecture, which is characterized at pre-weaned stages of development as having a poorly developed collagen matrix that can be easily disrupted.

The method of the invention stops the collagenase digestion of pancreatic tissue by adding an ice-cold buffered salt solution that is supplemented with either serum or albumin or both. In various embodiments, the method of the invention stops the collagenase digestion reaction by adding Hank's Balanced Salt Solution (HBSS)) supplemented with HEPES and 10% bovine serum albumin (BSA) to the reaction mixture. Following the stoppage of the collagenase digestion, the digested pancreatic tissue is filtered through a metal mesh funnel to remove debris and undesirably large tissue fragments. In various embodiments, the method of the invention uses a 500 μm metal mesh funnel, to remove tissue fragments that are greater than 500 μm.

As stated above, the method of the invention promotes maturation of islets. More particularly, the method of the invention comprises a period of tissue culture that is divided into at least two phases, a recovery phase and a maturation phase. These phases are distinguished by sequence (i.e., the recovery phase precedes the maturation phase) and by variations in concentrations of certain culture medium ingredients. The recovery phase of culturing islets commences after the collagenase digestion reaction that is described above is performed, and typically lasts about 48 hours. The maturation phase of culturing islets commences after the recovery phase, and typically lasts from about 6 to 8 days. Herein, it is to be understood that the terms “cell culture,” “islet culture,” and “culture,” refer to the maintenance of cells in an artificial, in vitro environment. The terms “islet” and “islet culture” refer to individual islet cells, e.g., beta cells, as well as collectively to all the cells that are contained in islets. The phrases “cell culture medium,” “culture medium,” “medium formulation,” or simply “medium” (plural “media” in each case) refer to a nutritive solution for cultivating cells, i.e., islets, and may be used interchangeably.

The recovery phase of the method of the invention relates to the initial 48 hour period of tissue culture of the filtered, collagenase digested pancreatic tissue that is described above. During the recovery period, the pancreatic tissue is cultured in a specialized medium that is referred to herein as “recovery maturation medium,” or as the “first maturation medium.” In general, recovery maturation medium is a basal medium that additionally comprises: a) an undefined component such as pig serum or extracts from animal embryos, organs or glands; b) an antioxidant compound; c) a component that enhances glucose-dependent insulin secretion by beta cells; d) a derivative of vitamin B; e) a derivative of vitamin E; f) heparin; g) a basic pancreatic trypsin inhibitor; h) a serine protease inhibitor; and i) a deoxyribonuclease.

In general, the basal medium can be any single basal medium solution or a combination of basal medium solutions that is compatible with islet cell maturation. For example, the basal medium may be selected from, but not limited to BME medium (Proc. Soc. Exp. Biol. Med., 89, 363 (1965)), BGJb medium (Exp. Cell Res., 25, 41 (1961)), CMRL 1066 medium (N.Y. Academy of Science, 5, 303 (1957)), Glasgow MEM medium (Virology, 16, 147 (1962)), Improved MEM Zinc Option medium (J. National Cancer Inst., 49, 1705 (1972)), IMDM medium (In Vitro, 9, 6 (1970)), Medium 199 medium (Proc. Soc. Exp. Biol. Med., 73, 1 (1950)), Eagle's MEM medium (Science, 130, 432 (1959)), Alpha MEM medium (Nature New Biology, 230, 310 (1971)), Dulbecco's MEM medium (Virology, 8, 396 (1959)), Ham's medium (Exp. Cell Res., 29, 515 (1963); Proc. Natl. Acad. Sci. USA, 53, 288 (1965)), RPMI 1640 medium (J. A. M. A., 199, 519 (1967)), Fischer's medium (Methods in Med. Res., 10 (1964)), McCoy's medium (Proc. Soc. Exp. Biol. Med., 100, 115 (1959)), William's E medium (Exp. Cell Res., 69, 106 (1971); Exp. Cell Res., 89, 139 (1974)), a mixed medium thereof and the like. In various embodiments of the invention, the basal medium of recovery maturation medium is Ham's F-12, while in other embodiments, it is Medium 199. In still other embodiments, the basal medium of recovery maturation medium is a mixture of Ham's F-12 and Medium 199 media. For example, the recovery maturation medium composition comprises Ham's F-12 and Medium 199 media mixed in a ratio that is selected from 1:10 to 10:1.

With respect to the undefined component of the recovery maturation medium, while pig serum, at a concentration of 1-20% v/v, is most commonly used as an undefined component, extracts from animal embryos, organs or glands (0.5-10% v/v). Other serum or albumin sources are also routinely used, including newborn calf, horse, and human. Organs or glands that have been used to prepare extracts for the supplementation of culture media include submaxillary gland (Cohen (1961) J. Biol. Chem. 237: 1555-1565), pituitary (Peehl and Ham (1980) In Vitro 16: 516-525; see U.S. Pat. No. 4,673,649), hypothalamus (Maciag, et al. (1979) Proc. Natl. Acad. Sci. USA 76: 5674-5678; Gilchrest, et al. (1984) J. Cell. Physiol. 120: 377-383), and brain (Maciag, et al. (1981) Science 211: 1452-1454). These types of chemically undefined supplements serve several useful functions in cell culture media (see Lambert, et al. (1985) In: Animal Cell Biotechnology, Vol. 1, Spier et al., Eds., Academic Press, New York, pp. 85-122 (1985)). For example, these supplements (1) provide carriers or chelators for labile or water-insoluble nutrients; (2) bind and neutralize toxic moieties; (3) provide hormones and growth factors, protease inhibitors and essential, often unidentified or undefined low molecular weight nutrients; and (4) protect cells from physical stress and damage. Thus, serum or organ/gland extracts are commonly used as relatively low-cost supplements to provide an optimal culture medium for the cultivation of animal cells.

With respect to the antioxidant compound component of the recovery maturation medium, this component functions to prevent damage to important cellular components caused by reactive oxygen species such as free radicals and peroxides. In various embodiments, the antioxidant component is glutathione. The amount of glutathione present in recovery maturation medium may vary, but it is generally present at concentrations that fall within the range of 0.0025-0.125% (w/v).

As stated above, the recovery maturation medium comprises a component that enhances glucose-dependent insulin secretion by beta cells. In various embodiments, this component is a synthetic or natural peptide hormones that exert potent glucoregulatory action through their glucose-dependant stimulation of insulin secretion. Examples of such peptides include, but are not limited to, gastric inhibitory polypeptide (GIP) and glucagon-like peptide 1 (GLP-1), as well as mimetics and analogs of these peptides. In various embodiments of the invention, the recovery maturation medium comprises the GLP-1 mimetic, Exenatide™ (a synthetic form of exendin-4 manufactured by California Peptide Research, Inc., Napa, Calif.) at sufficient concentrations to promote beta cell maturation. In other embodiments of the invention, a component that enhances glucose-dependent insulin secretion by beta cells is be insulin or an insulin derivative. The insulin can be derived from any species such as human, bovine, porcine, equine, canine or murine, as well as be a synthetic insulin or an insulin analog. The term “insulin analog” and the like are used interchangeably herein and are intended to encompass any form of “insulin” as defined above, wherein one or more of the amino acids within the polypeptide chain has been replaced with an alternative amino acid and/or wherein one or more of the amino acids has been deleted or wherein one or more additional amino acids has been added to the polypeptide chain or amino acid sequences, which still has at least one function of native insulin such as for example, decreasing blood glucose levels. In certain embodiments, the recovery maturation medium can include insulin-transferrin-sodium selenite (ITS) hormone. In still other embodiments, the component of recovery maturation medium that enhances glucose-dependent insulin secretion by beta cells can be any combination of the aforementioned factors.

Derivatives of the vitamin B and E families are also added to the recovery maturation medium to help in certain energy formation components and to reduce oxidative stress or damage. While any derivative of vitamin B may be appropriate, examples of vitamin B derivatives include, but are not limited to nicotinamide, nicotinamide analogs, a nicotinamide or a nicotinamide analog derivative, or a nicotinamide or a nicotinamide analog metabolite. In various embodiments, the vitamin b derivative is present in the recovery maturation medium at concentrations that can range from 0.02-0.12% (w/v). The derivatives of vitamin E that can be added to the recovery maturation medium of the invention are any that are known to those of ordinary skill in the art, including, but not limited to vitamin E (α-tocopherol), α-tocopherol succinate, α-tocopherol phosphate, Trolox™ (6-hydroxy-2,5,7,8-tetramethylchroman-2-carboxylic acid), and vitamin E itself, as well as any combinations thereof. In various embodiments, the vitamin E derivative component of recovery maturation medium is present at concentrations that range from 1.0×10⁻⁴-5×10-⁴% (w/v).

With respect to the basic pancreatic trypsin inhibitor component of the recovery maturation medium, suitable inhibitors include, but are not limited to inhibitors of luminally secreted proteases, such as aprotinin, Bowman-Birk inhibitor, soybean trypsin inhibitor, chicken ovomucoid, chicken ovoinhibitor, human pancreatic trypsin inhibitor, camostate mesilate, flavonoid inhibitors, antipain, leupeptin, p-aminobenzamidine, AEBSF, TLCK, APMSF, DFP, PMSF, poly(acrylate) derivatives, chymostatin, benzyloxycarbonyl-Pro-Phe-CHO, FK-448, sugar biphenylboronic acids complexes, .beta.-phenylpropionate, elastatinal, methoxysuccinyl-Ala-Ala-Pro-Val-chloromethylketone (MPCMK), EDTA, and chitosan-EDTA conjugates. Suitable protease inhibitors also include inhibitors of membrane bound proteases, such as amino acids, di- and tripeptides, amastatin, bestatin, puromycin, bacitracin, phosphinic acid dipeptide analogues, .alpha.-aminoboronic acid derivatives, Na-glycocholate, 1,10-phenanthroline, acivicin, L-serine-borate, thiorphan, and phosphoramidon. In various embodiments, the basic pancreatic trypsin inhibitor component of the recovery maturation medium is present at concentrations of about 20 to 500 mM.

As stated above, the recovery maturation medium can comprise heparin. Typically, heparin is added to the medium in the form of heparin sodium. In various embodiments, the heparin component of the recovery maturation medium of the invention can be heparan sulfate, present either in place of, or in combination with heparin.

As stated above, the recovery maturation medium can comprise a serine protease inhibitor component. In various embodiments, a serine protease inhibitor will inhibit the activation of endogenous enzymes of the donor pancreas(es) that can be activated during the collagenase digestion step of the islet isolation procedure that is described above. See Rose N L et al. (2003) Transplantation 75(4):462-466, which is incorporated herein. In various embodiments, the recovery maturation medium of the invention comprises the protease inhibitor, Pefabloc™ (Roche Biochemicals Inc., Indianapolis, Ind.). In various other embodiments, the serine protease inhibitor component of the recovery maturation medium is present at concentrations that range from 0.2 to 0.7 mM.

With respect to the DNase component of the recovery maturation medium of the invention, this component is present in the medium to prevent cell clumping which tends to happen when cells die and release deoxyribonucleic acid (DNA). Examples of suitable DNases include the Dnase I as well as Dnase II forms of DNas, including recombinant forms of these enzymes that are derived from any mammalian source, including human, bovine and porcine DNases. In various embodiments, the DNase component of the recovery maturation medium is Dornase alfa, a recombinant human form of DNase I that is commercially available as Pulmozyme™ (Genentech, South San Francisco, Calif.). In various other embodiments, the DNase component of the recovery maturation medium can be present at concentrations that range from 200 to 400 mM.

The recovery maturation medium can also optionally comprise an antibiotic or combination of antibiotics. Ideally, antibiotics that are considered for inclusion in media of the invention are stable across a broad range of temperatures, and are neither inactivated by proteins nor inhibit protein moieties in a manner that would interfere with islet growth or maturation. In various embodiments of the invention, recovery maturation medium comprises gentamycin sulfate at a standard concentration for its use in tissue culture, e.g., 0.0025% (w/v).

As stated above, the method of the invention comprises a period of tissue culture that is divided into at least two phases, a recovery phase and a maturation phase. The maturation phase generally begins immediately after the 48 recovery phase, and involves replacing the islet culture's recovery maturation medium with “maturation medium,” or as the “second maturation medium.” Maturation medium has an identical composition to recovery maturation medium, with the exception that maturation medium contains one-half the amounts of the serine protease inhibitor and a deoxyribonuclease components. The method of the invention replaces 50% of the maturation medium at least every 48 hours during the period of beta cell maturation. At each 48 hour timepoint, the number of islets are counted and checked for viability. In various embodiments, the method of the invention determines the islet count (IC) and the islet equivalence (IE) by staining an aliquot of the culture with Dithizone (DTZ), which binds zinc ions present in the islets' beta cells, and therefore stains the islets red. In more specific embodiments, the method of the invention removes a 100 μl aliquot of the pancreatic tissue culture, and mixes the aliquot with 1 ml of DTZ. Analysis of the DTZ-stained cells by either confocal microscopy or flow cytometry identifies mature beta cells. Thus, DTZ staining allows a determination of the proportion of cells in the islet that are mature beta cells. In various embodiments the method of the invention yields about 1×10² or more IE per gram of pancreatic tissue. For example, the method of the invention may yield, but is not limited to, 5×10², 1×10³, 5×10³, 1×10⁴, 5×10⁴, or any IE yield therein, IE per gram of pancreatic tissue. By way of example, in certain embodiments, the method of the invention yields can be 6.4×10³ IE per gram of harvested pancreas prior to processing at eight days post islet isolation.

Viability of the islet cultures during the beta cell maturation period can generally be determined by the method of the invention by staining an aliquot of an islet culture with either fluorescein diacetate (FDA), propidium iodide (PI), or Newport Green, and then analyzing the stained cells by either confocal microscopy, or by using a microplate reader. In various embodiments the method of the invention yields cell viabilities of greater than 80% at seven days post islet isolation. For example, the cell viability of an islet culture at seven days post islet isolation can be, but is not limited to, 85%, 90%, 95%, 100%, or any percentage of cell viability therein at seven days post islet isolation. In certain embodiments, for example, cell viability can be greater than 98% at seven days post islet isolation according to the FDA and PI methods of assessing viability. In certain other embodiments, cell viability can be greater than 90% at seven days post islet isolation according to the Newport Green method of assessing viability.

One of the important functions of a beta cell is to adjust its insulin secretion according to the glucose level. With that function in mind, the glucose responsiveness of the islet cultures can monitored over the maturation period of the method of the invention by using any method known by one of skill in the art to be suitable for this purpose. For example, a glucose stimulated insulin release (GSIR) assay can be used to assess islet function at any stage of beta cell maturation process described herein. More particularly, a standard GSIR assay incubates approximately between 100 to 150 islet cells obtained from the islet cultures in the following order of solutions for specified periods of time, generally one hour, with two PBS washes with PBS between each glucose solution incubation. The assay then detects the amount of insulin secreted by the cells: 1) a low glucose solution (2.8 mM glucose); 2) a high glucose solution (28 mM glucose); 3) high glucose solution (28 mM glucose) that also contains 3-isobutyl-1-methylxanthine (IBMX, 50 μM); and then 4) low glucose solution (2.8 mM glucose).

The insulin release data is then used to calculate a stimulated index (SI) based on the ratio of insulin secreted by beta cells under high glucose conditions to the amount of insulin secreted by beta cells under low glucose conditions. A maximum SI (MX) can be calculated as the amount of insulin secreted under high glucose plus IBMX conditions divided by the amount of insulin secreted under merely high glucose conditions.

To detect secreted insulin, The medium is aspirated from the plates, spun, and is collected separately after each of the glucose simulation steps described above. The supernatants are collected for insulin analysis, and stored at −20° C. until insulin content is determined by any method for detecting insulin that is known in the art and that is appropriate for this purpose in the method of the invention. In various embodiments, the GSIR assay of the invention detects insulin by a radioimmunoassay (RIA), while in other embodiments, the assay uses a standard enzyme-linked-immunosorbent-assay (ELISA) to detect insulin.

In general, the islets that are isolated by the method of the invention are glucose responsiveness immediately after their isolation, a stage at which many of the beta cells are immature. As beta cells mature under the method of the invention, sequential increases in glucose responsiveness can be observed. In various embodiments, the method of the invention increases the SI of the islet culture over time. In certain embodiments, the increase in SI may be about 5% or less between timepoints during the beta cell maturation process, whereas in other embodiments, the percentage increase in SI may range from, and including, 5% to 200%. For example, in some embodiments, the SI may be about 1.3 at the time of isolation, 1.7 at day 3 post-isolation, and about 2.6 at day 7 post-isolation.

As stated above, during the beta cell maturation process of the method of the invention, the cultured islets undergo certain developmental changes, which are evidenced by the display of various phenotypic and genotypic indicia of differentiated pancreatic cells. Indeed, there are a number of cellular markers that can be used to identify populations of pancreatic cells. It is believed that the changes in these indicia or markers relate to the maturation of the cultured islets. Among these changes, inter alia, are a decrease in the proportion of cells that are apoptotic, the expression of cellular markers of beta cell maturation, and an increase in the size of islets.

Changes in the level of apoptosis during the beta cell maturation period of the method of the invention can be determined by using any method known by one of skill in the art to be suitable for this purpose, including using commercially available apoptosis detection kits. Other exemplary methods for detecting apoptosis in islet cultures include carbocyanine nucleic acid stain-based techniques, such as the YO-PRO®-1 Iodide and Vybrant® Apoptosis Assay Kits (both from Life Technologies). Generally, islets undergo a progressive decrease in apoptosis during the beta cell maturation process, primarily in the outer acinar cell layers of the islets. In various embodiments, the portion of islet cells undergoing apoptosis can decrease ten-fold or more over the course of the beta cell maturation process. For example, the portion of apoptotic islet cells at day 3 post-isolation may be about 22.5%, then decrease to about 4.2% by day 8 post-isolation.

Cellular markers of beta cell maturation can be detected by using any method known by one of skill in the art to be suitable for this purpose. For example, one standard method of detecting islet cell markers is by FLOW cytometry. Briefly, filtered (e.g., a 70 μm filter), dispase-dissociated islet cell suspensions can be immunohistochemically stained with antibodies that are specific for insulin (specific for beta cells), glucagon (specific for alpha cells), somatostain (specific for delta cells), pancreatic polypeptide (pp) (specific for pp-producing cells) and Amylase (specific for acinar tissue). In various embodiments of the invention, the proportion of islet cells that stain positively for Amylase,(i.e., acinar cells), decreases, while the proportion of islet cells that stain positively for insulin (i.e., beta cells increases over time. In certain embodiments the proportion of Amylase positive cells at day 2 versus day 7 post-isolation may decrease from about 75% to 10%, respectively; for example, from about 65% to about 25%. Conversely, in certain embodiments, the proportion of insulin-positive cells at day 2 versus day 7 post-isolation may increase from about 25% to about 85%, respectively; for example, from about 42% to about 70%.

Islet cell morphology and architecture can be determined by using any method known by one of skill in the art to be suitable for this purpose. Generally, standard hematoxylin and eosin staining (H&E) staining of islets reveals their cellular composition and structural characteristics. As stated above, the diameter of an islet correlates positively to functionality, (i.e., glucose responsiveness). In that regard, the method of the invention can result in cultures at day 8 post-isolation in which a significant majority of the islets are more than 50 μm in diameter. For example, in various embodiments, the method of the invention yields islet cultures in which the diameters of about 90% or more of the islets are between, and including, 50 μm to 150 μm.

### EXAMPLES

### Example 1

### Islet Removal and Isolation

Weanling piglets were used as the donor source of immature islets. A typical islet preparation procedure used ten 14 to 26 days old Yorkshire piglets. The pancreases were removed from the piglets by rapid surgical procurement, i.e., less than five minutes, and then placed in ice-cold Organ Preservation Solution (Corning Cellgro). The total cold ischemia time was limited to 20 minutes. The harvested pancreases were pooled and sectioned as follows. While chilled, pancreases were trimmed of surrounding adipose and lymphatic tissue, and then finely minced until the tissue sections were generally 0.5 mm to 1.0 mm in diameter. The sectioning process was performed using standard scissors and blender technology to mince the pancreatic tissue. The minced tissue was added to a low dose Clzyme collagenase MA/BP protease enzyme mixture (75-250 mg/pancreas, VitaCyte LLC, Indianapolis, Ind.) and allowed to digest at 37° C. and gentle rotary shaking (40-60 rmp) until the majority of surrounding acinar tissue gently separated from the immature islets. The pancreatic tissue was then allowed to digest for another 150 minutes under the same condition. At the end of the digestion reaction, 100-150 ml of cold Hank's Balanced Salt Solution (HBSS) supplemented with HEPES and 10% BSA was added to the tissue-collagenase mixture to stop the digestion reaction. The digestion time was kept as brief as possible in order to help preserve the nascent islet architecture because the collagen matrix of the islets at the time of their isolation is poorly developed and subject to easy disruption. Digested tissue was filtered through a 500 μm metal mesh funnel, to remove undigested tissue greater than 500 μm in diameter, leaving immature islets were generally between 50 μm and 500 μm in diameter. The islets were then subjected to tissue culture conditions that allowed them to undergo maturation. Briefly, the culturing of islets was divided into two phases: i) a recovery phase; and ii) a maturation phase. The recovery phase related to the 48 hour period following the surgical isolation of islets, and the maturation phase related to period from the end of the recovery phase to the time at which the islets are sufficiently mature to be glucose responsive within one day of being xenotransplanted, typically about eight days. These culturing phases are described in the examples below.

### Example 2

### Recovery of Isolated Islets

For the first 48 hours post-isolation, islet clusters were cultured in a novel tissue culture medium that is termed recovery maturation medium. The first step in the preparation of recovery maturation medium involved obtaining a Ham's F-12/Medium 199 basal medium (F-12/199 medium) in which the Ham's F-12 and Medium 199 components are present in a 1:1 ratio. In these studies, F-12/199 medium was prepared by adding Ham's F-12 and Medium 199 powders (both purchased from Cellgro, Inc.) in sterile water that met ISO 9001:2000 and cGMP guidelines (Thermo Scientific Inc., Waltham, Mass.) at weight/volume (w/v) concentrations of 0.815% each (i.e., 8.15 g in 1 L water). The F-12/199 medium was then supplemented with: 5.5% (w/v) porcine serum (Lampire Biological Laboratories, Inc., Pipersville, Pa.); 2.5×10⁻³% (w/v) gentamycin sulfate (Fisher Bioreagents, Thermo Fisher Scientific, Inc., Waltham, Mass.); 0.062% (w/v) glutathione tripeptide (Acros Organics, Thermo Fisher Scientific, Inc., Waltham, Mass.); 0.6% (w/v) Insulin-transferrin-sodium selenite (ITS) hormone (Sigma-Aldrich Co., LLC, St. Louis, Mo.); 0.029% (w/v) L-glutamine (Fisher Bioreagents, Thermo Fisher Scientific, Inc., Waltham, Mass.); 0.0244% (w/v) 6-Hydroxy-2,5,7,8-tetramethylchroman-2-carboxylic acid (Trolox™, Acros Organics, Thermo Fisher Scientific, Inc., Waltham, Mass.); 4.2×10⁻⁶% (w/v) Exenatide (a synthetic form of exendin-4 manufactured by California Peptide Research, Inc., Napa, Calif.), 0.066% heparin (150 U/mg heparin sodium, Fisher Bioreagents, Thermo Fisher Scientific, Inc., Waltham, Mass.); 215 mM Aprotinin (Sigma-Aldrich Co., LLC, St. Louis, Mo.); 0.5 mM Pefabloc™ (Sigma-Aldrich Co., LLC, St. Louis, Mo.); 417 mM Dnase Alfa (Genentech Inc., S. San Francisco, Calif.); and a sufficient amount of HEPES buffer to maintain the pH of the medium at 7.2 to 7.5 (typically from 5 mM to 20 mM). The islets were cultured in the recovery maturation medium at 37° C. and 5% CO₂ in T-175 suspension flasks at a tissue density of 80-100 μl of tissue in 40 ml of culture media.

### Example 3

### Maturation of Excised Islets

After 48 hours of being cultured in recovery maturation medium, the islets were removed from the recovery maturation medium, centrifuged for two minutes at 200×g, and re-suspended in a modified version of the recovery maturation medium that is referred to herein simply as “maturation medium.” Specifically, the difference between recovery maturation medium and maturation medium is that maturation medium has one-half the amounts of Pefabloc™ and DNase Alfa. The islets were cultured in maturation medium until the islets matured fully, a period of time of about eight to nine days, during which 50% medium replacements were made every 48 hours. Full maturation of islets was defined in accordance with the histochemical and functional analyses described in the following examples. When the islets reached full maturation, they could be cultured long-term in a “supplemented maturation medium,” which was maturation medium that contained an additional 5 mM concentration of calcium. The extra calcium was required to help prevent the disintegration of the islet capsules.

### Example 4

### Islet Viability, Number and Maturity

Aliquots of tissue were evaluated for islet counts, cell viability, and beta cell maturity every 48 hours when the medium was replaced. The number of islets in the culture, referred to herein as the islet count (IC), and the number of islets containing maturing beta cells, referred to herein as the islet equivalence (IE), were determined by adding a 100 μl aliquot of islet culture to 1 ml Dithizone (DTZ), a red stain that binds zinc ions present beta cells (Latif Z A, et al. (1988) Transplantation 45(4):827-830; Ricordi C, et al. (1990) Acta Diabetol Lat 27(3):185-95; and Scharp D W, et al. (1984) World J Surg. 8(2):143-151). After allowing DTZ staining to proceed for 5 minutes, the islets were observed at 25× magnification on a standard stereomicroscope (Max Erb Instrument Co., Santa Ynez, Calif.) that was equipped with a 10×10 eye piece graticule. Each islet cluster was counted to arrive at the IC. Positive DTZ staining was used to determine percentage of islet purity over the maturation period, based on the detected presence of maturing beta cells. More particularly, percent purity was determined by amount of positive DTZ staining divided by the amount of unstained regions within each cluster of tissue.

Percent cell viability was analyzed by using either fluorescein diacetate (FDA) and propidium iodine (PI) or Newport Green/PI staining followed by visualization by confocal microscopy using a Zeiss LSM 510 confocal microscope (Carl Zeiss, Inc.), and viability was quantified with a Microplate reader (Tecan Infinite F200, Magellan V7) (Iglesias I, et al. (2008) Transplant Proc. 40(2):351-354; Lamb M, et al. (2011) Transplant Proc. 43(9):3265-3266; and Lukowiak B, et al. (2001) J Histochem Cytochem. 49(4):519-528).

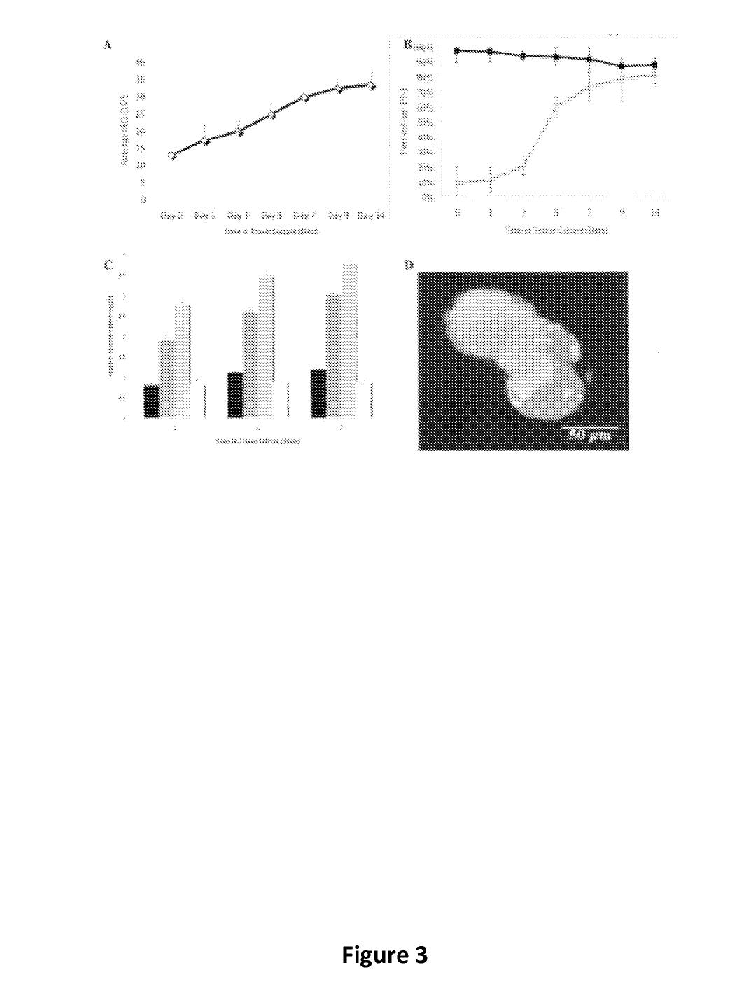

Immediately following their isolation, islets were, on average, 8.5%±1.1 pure. See FIG. 3B. After 7 days of culturing, the purity of the islets had increased to 78%±1.5. See FIG. 3B. The proportion of DTZ positive tissue increased during tissue culture (12.6×10³±183 IEQ (mean±sem) to 33.3×10³±136 after 7 days of culture (n=10), p<0.05)) with a majority of islets between 50-150 μm in diameter (94.52±11% after 7 days). See Table 1 and FIG. 3A.

Full maturation of the cultured islets occurred by 7 days post isolation with no islets that exceeded 350 μm in diameter, and approximately 90% of islets were between 50 μm and 150 μm. See Table 1. Viability was >98%±0.03 (FDA/PI) and >90%±0.4 (Newport Green) at day 7 of culture. See FIG. 3B.

TABLE 1

| | | Islet Parameters | | | | | | | | |
|---|---|---|---|---|---|---|---|---|---|---|
| Culture | | | | Purity | Islet Size Distribution (%) | | | | | |
| Period | n | IC (10³) | IE (10³) | (%) | 50-100 μm | 100-150 μm | 150-200 μm | 200-250 μm | 250-300 μm | 300-350 μm |
| 0 days | 16 | 10.4 ± 3.4 | 12.6 ± 2.1 | 8.5 ± 1.1 | 71.70 ± 5 | 26.42 ± 8 | 1.89 ± 4 | 0.00 | 0.00 | 0.00 |
| 3 days | 16 | 24.8 ± 8.2 | 19.7 ± 3.3 | 20 ± 0.6 | 51.16 ± 8 | 27.91 ± 6 | 11.63 ± 2 | 6.98 ± 3 | 2.33 ± 1 | 0.00 |
| 7 days | 20 | 32.0 ± 5.8 | 33.3 ± 6.4 | 78 ± 1.5 | 49.32 ± 9 | 34.25 ± 4 | 10.96 ± 5 | 2.74 ± 2 | 2.74 ± 3 | 0.00 |
| 14 days | 10 | 24.0 ± 4.9 | 32.1 ± 6.6 | 81 ± 2.5 | 44.44 ± 3 | 33.33 ± 2 | 11.11 ± 4 | 8.89 ± 6 | 2.22 ± 1 | 0.00 |

### Example

### Islet Function

Islet function during the maturation process was determined every 48 hours by assessing glucose mediated insulin release (GSIR). At each time point, islets were incubated for 1 hour, in the following order of solutions: 1) low glucose (2.8 mM); 2) high glucose (28 mM); 3) high glucose (28 mM) plus 3-isobutyl-1-methylxanthine (IBMX, 50 μM); and then 4) low glucose (2.8 mM). A stimulated index (SI) was calculated as the ratio of insulin secreted in high glucose over the amount of insulin in low glucose. The maximum stimulated index (MX) was calculated as the amount of insulin secreted in high glucose plus IBMX divided by the amount of insulin secreted in high glucose. Islets were glucose responsiveness was immediately after their isolation. Sequential increases in glucose responsiveness occurred at culture days 3, 5 and 7, respectively. See FIG. 3C. Islet function (i.e., GSIR) improved during time in culture, as observed by the increase in the value of the SI from day 0 (1.3±0.1) to day 7 (2.6±0.2) of tissue culture.

### Example

### Apoptosis in Islets

Islets underwent a progressive decrease in apoptosis during the maturation process, primarily in the outer acinar cell layers of the islets. See FIG. 3(D). Overall, the portion of apoptotic cells in the islets was 22.5%±1.0 on at day 3, and 4.2%±0.5 at day 8.

### Example

### Islet Cellular Composition

To characterize cellular composition of islets of the course of their maturation in tissue culture, groups of 15,000-20,000 islet equivalents (IE) from day 0 (first day of recovery phase culture), day 3, day 7, and day 11, were twice washed and centrifuged at 200×g in phosphate buffered saline (PBS), and then subjected to a 15-minute Acutase™ (500-720 U/ml, Innovative Cell Technologies) dissociation reaction at 37° C. that was performed according to the manufacturer's instructions. Single cell suspensions of the islet cells were then filtered through a 40 μm filter to remove debris, and then the cells were stained with antibodies that were specific for: i) the acinar cell marker, amylase (a sheep polyclonal Anti-amylase purchased from Abcam, PLC, Cambridge, Mass.); ii) the beta cell marker, c-peptide (a mouse monoclonal anti-C-peptide purchased from Abcam, PLC); and iii) the alpha cell marker glucagon (a rabbit polyclonal anti-glucagon purchased from Abcam, PLC) for 15 minutes. The cells were then twice washed in DPBS, and centrifuged at 1500×g for 5 minutes followed by incubation with corresponding secondary antibodies for 15 minutes. (Alexa™ 555 Goat anti-mouse, Alexa™ 488 Goat anti-Rabbit, DyLight™ 650 Dnk aAb to sheep, which were respectively purchased from Life Technologies Corp., Carlsbad, Calif.; Life Technologies Corp.; and Abcam PLC). Th cell suspension was then stained for viability using Propidium iodine (PI) for 15 minutes. Cell populations were then quantified using flow cytometery (BD FACS Aria™ II, FACS Diva™ software V. 6.5) by running samples in triplicate through a 100 um nozzle at 25 psi. The results from these analyses are reported in Table 2. Results are given as mean±sem, n=3, performed in triplicate. Amylase proportions decreased from 63.9±10% at day 0 to 20.4±6% at day 7 of culture, while the beta cell proportion within the islets increased from 16.9±3% to 25.4±4% and 49.8±7% after 3 and 7 days in culture, respectively (p<0.05). After 11 days of culture, beta cell populations further increased to 54.4±8%, and the proportion of acinar cells further decreased to 13.0±8%. No significant difference in glucagon positive cells was observed during over the course of culture from day 3 to day 11 (30.5±6% at day 3 vs. 27.6±3% at day 7 and 30.7±6% at day 11, p=ns).

TABLE 2

| Days Post | % Beta Cells | % Alpha Cells | % Acinar Cells | % Viable |
|---|---|---|---|---|
| Digestion | (C-Peptide) | (Glucagon) | (Amylase) | (PI) |
| 0 | 16.9 ± 3% | 17.0 ± 2% | 63.9 ± 10% | 80 ± 5% |
| 3 | 25.4 ± 4% | 30.5 ± 6% | 44.0 ± 4% | 75 ± 12% |
| 7 | 49.8 ± 7% | 27.6 ± 3% | 20.4 ± 6% | 89 ± 3% |
| 11 | 54.4 ± 8% | 30.7 ± 6% | 13.7 ± 8% | 85 ± 8% |

1. A method for promoting the maturation of islet cells from a pre-weaned mammal, comprising:
a) removing a pancreas from a pre-weaned mammal;
b) reducing the pancreas tissue to fragments that are greater than the size of an intact islet while retaining islets in their whole, insulin-producing condition;
c) digesting the tissue fragments in a protease solution such that any exocrine tissue that surrounds the islets is only partially separated from the islets;
d) culturing the digested tissue in a first maturation medium;
e) culturing the digested tissue in a second maturation medium;
f) determining the glucose responsiveness of the cultured islets; and
g) selecting glucose-responsive islets.

2. The method of claim 1, wherein the pre-weaned mammal is a piglet.

3. The method of claim 1, wherein the first maturation medium comprises:
a) an undefined component of animal origin; b) an antioxidant compound; c) a component that enhances glucose-dependent insulin secretion by beta cells; d) a derivative of vitamin B; f) a derivative of vitamin E; g) heparin; h) a basic pancreatic trypsin inhibitor; i) a serine protease inhibitor; and j) a deoxyribonuclease

4. The method of claim 1, wherein the second maturation medium comprises:
a) an undefined component of animal origin; b) an antioxidant compound; c) a component that enhances glucose-dependent insulin secretion by beta cells; d) a derivative of vitamin B; f) a derivative of vitamin E; g) heparin; h) a basic pancreatic trypsin inhibitor; i) a serine protease inhibitor; and j) a deoxyribonuclease, wherein the concentrations of the serine protease inhibitor and deoxyribonuclease components are no more than one-half of their concentrations in the first maturation medium.

5. The method of claim 3, wherein the undefined component of animal origin is pig serum.

6. The method of claim 3, wherein the antioxidant compound is glutathione.

7. The method of claim 3, wherein the component that enhances glucose-dependent insulin secretion by beta cells is either Exenatide™, insulin, insulin-transferrin-sodium selenite (ITS) hormone, or a combination thereof.

8. The method of claim 3, wherein the derivative of vitamin B is nicotinamide.

9. The method of claim 3, wherein the derivative of vitamin E is 6-Hydroxy-2,5,7,8-tetramethylchroman-2-carboxylic acid.

10. The method of claim 3, wherein the basic pancreatic trypsin inhibitor is aprotinin.

11. The method of claim 3, wherein the serine protease inhibitor is Pefabloc™.

12. The method of claim 3, wherein the deoxyribonuclease is dornase alfa.

13. The method of claim 4, wherein the undefined component of animal origin is pig serum.

14. The method of claim 4, wherein the antioxidant compound is glutathione.

15. The method of claim 4, wherein the component that enhances glucose-dependent insulin secretion by beta cells is either Exenatide™, insulin, insulin-transferrin-sodium selenite (ITS) hormone, or a combination thereof.

16. The method of claim 4, wherein the derivative of vitamin B is nicotinamide.

17. The method of claim 4, wherein the derivative of vitamin E is 6-Hydroxy-2,5,7,8-tetramethylchroman-2-carboxylic acid.

18. The method of claim 4, wherein the basic pancreatic trypsin inhibitor is aprotinin.

19. The method of claim 4, wherein the serine protease inhibitor is Pefabloc™.

20. The method of claim 4, wherein the deoxyribonuclease is dornase alfa.
